(19) United States
(12) Patent Application Publication
(10) Pub. No.: US 2020/0197211 A1
(43) Pub. Date: Jun. 25, 2020

(54) DEFORMED NAIL CORRECTOR

(71) Applicants: MARUHO HATSUJYO KOGYO CO., LTD., Kyoto (JP); MARUHO CO., LTD., Osaka (JP)

(72) Inventors: Yasuyuki HITOMI, Kyoto (JP); Soki TAKINO, Kyoto (JP); Yoshihiro NAKAHASHI, Osaka (JP); Noriko HONDA, Osaka (JP)

(73) Assignees: MARUHO HATSUJYO KOGYO CO., LTD., Kyoto (JP); MARUHO CO., LTD., Osaka (JP)

(21) Appl. No.: 16/616,647

(22) PCT Filed: Nov. 14, 2017
(86) PCT No.: PCT/JP2017/040988

§ 371 Date: Feb. 21, 2020

(30) Foreign Application Priority Data
May 26, 2017 (JP) 2017-104167

(51) Int. Cl. A61F 5/11

(52) U.S. Cl. CPC A61F 5/11

(57) ABSTRACT

A deformed nail corrector 1A for correcting a deformed nail includes a first hook 2, a second hook 3, a tube-like flexible member 4 joined at one end to the first hook 2 and at the other end to the second hook 3, and a superelastic wire member 5 inserted in the flexible member 4, and the flexible member 4 is stretchable so as to allow the distance between the first hook 2 and second hook 3 to be changed.

This nonprovisional application is a National Stage of International Application No. PCT/JP2017/040988, which was filed on Nov. 14, 2017, and which claims priority to Japanese Patent Application No. 2017-104167, which was filed in Japan on May 26, 2017, and which are both herein incorporated by reference.

### TECHNICAL FIELD

The present invention relates to a deformed nail corrector for correcting a deformed nail such as a pincer nail or an ingrown nail.

### BACKGROUND ART

For example, a conventionally known deformed nail corrector includes two elastic plates welded to L-shaped crossbars and threads for binding the two elastic plates (see, for example, Patent Document 1).

When this deformed nail corrector is attached to a deformed nail, the two elastic plates are bent along the deformed nail, thereby creating restoring forces (i.e., forces that cause the plates to return to original shapes). By maintaining the restoring forces for a long period, the deformed nail can be corrected.

### PRIOR ART DOCUMENT

### Patent Document

Patent Document 1: Japanese Laid-Open Patent Publication No. 2011-98081

### DISCLOSURE OF THE INVENTION

### Problems to be Solved by the Invention

Incidentally, in an attachment method for the conventional deformed nail corrector, initially, a hook of a first elastic plate is attached to one edge of a deformed nail in the width direction, and a hook of a second elastic plate is attached to the other edge of the deformed nail in the width direction. Next, the first elastic plate is fitted under a crossbar of the second elastic plate, and the second elastic plate is fitted under a crossbar of the first elastic plate. Lastly, threads are engaged with the respective crossbars so as to bind the two elastic plates. In this manner, the conventional deformed nail corrector requires complex and troublesome attachment work.

The present invention has been achieved under the above circumstances, with a problem thereof being to provide a deformed nail corrector that is readily attachable.

### Solution to the Problems

To solve the above problem, the present invention provides a deformed nail corrector for correcting a deformed nail, including a first hook attachable to a first edge of the deformed nail in a width direction, a second hook attachable to a second edge of the deformed nail in the width direction, a tube-like flexible member joined at a first end to the first hook and at a second end to the second hook, and a superelastic wire member inserted in the flexible member, wherein the flexible member is stretchable so as to allow a distance between the first hook and the second hook to be changed.

In the deformed nail corrector, the flexible member may be joined to the first hook having an end inserted from the first end of the flexible member, and also joined to the second hook having an end inserted from the second end of the flexible member.

In the deformed nail corrector, when the first hook has a hollow shape with an opening at the end and the second hook has a hollow shape with an opening at the end, the wire member preferably has a first end slidably inserted in the first hook and a second end slidably inserted in the second hook.

In the deformed nail corrector, when the flexible member is a coil including a first welded portion, a second welded portion, and an intermediate portion between the first welded portion and the second welded portion, the first welded portion has a welded outer surface at the first end, and the second welded portion has a welded outer surface at the second end, the first welded portion and the second welded portion are preferably more rigid than the intermediate portion, and the wire member preferably has a first end positioned within an area of the first welded portion and a second end positioned within an area of the second welded portion.

In the deformed nail corrector, when the flexible member is a helical tube including a first rigid portion at the first end, a second rigid portion at the second end, and an intermediate portion with a helical cut, the first rigid portion and the second rigid portion are preferably more rigid than the intermediate portion, and the wire member preferably has a first end positioned within an area of the first rigid portion and a second end positioned within an area of the second rigid portion.

In the deformed nail corrector, the first hook and the second hook may include barbed portions at tips.

In the deformed nail corrector, the wire member may have an arc-like shape and tend to return to the arc-like shape when deformed.

In the deformed nail corrector, the flexible member may have an outer surface joined to the first hook at the first end and to the second hook at the second end, so as to allow the wire member to be put in and out of the flexible member from either the first or second end, or both.

In the deformed nail corrector, the first hook preferably has a first main hook portion and a first extended portion formed on a top surface of the first main hook portion, the first extended portion preferably has a first surface extending upward from the top surface, the second hook preferably has a second main hook portion and a second extended portion formed on a top surface of the second main hook portion, the second extended portion preferably has a second surface extending upward from the top surface, and the flexible member preferably has a bottom portion joined at the first end to the top surface of the first main hook portion and at the second end to the top surface of the second main hook portion, such that the opening at the first end is opposed to the first surface and the opening at the second end is opposed to the second surface.

In the deformed nail corrector, the wire member is preferably longer than a natural length of the flexible member, the first extended portion preferably has a first groove formed in the first surface so as to accommodate a first end of the wire member, and the second extended portion preferably has a second groove formed in the second surface so as to accommodate a second end of the wire member.

### Effect of the Invention

The present invention renders it possible to provide a deformed nail corrector that is readily attachable.

### BRIEF DESCRIPTION OF THE DRAWINGS

FIG. 1A provides a view illustrating a deformed nail corrector according to a first embodiment in non-attached state.

FIG. 1B provides a view illustrating the deformed nail corrector according to the first embodiment in attached state.

FIG. 2 is a view illustrating the structure of the deformed nail corrector according to the first embodiment.

FIG. 3 is a view illustrating the structure of a deformed nail corrector according to a second embodiment.

FIG. 4 is a view illustrating the structure of a deformed nail corrector according to a third embodiment.

FIG. 5 is a view illustrating the structure of a deformed nail corrector according to a fourth embodiment.

FIG. 6 is a view illustrating the structure of a deformed nail corrector according to a fifth embodiment.

FIG. 7 is a view illustrating the structure of a deformed nail corrector according to a sixth embodiment.

FIG. 8 is a view illustrating the structure of a deformed nail corrector according to a seventh embodiment.

FIG. 9 is a view illustrating the structure of a deformed nail corrector according to an eighth embodiment.

FIG. 10 is a view illustrating the structure of a deformed nail corrector according to a ninth embodiment.

FIG. 11 is an oblique view illustrating the deformed nail corrector according to the ninth embodiment.

### MODE FOR CARRYING OUT THE INVENTION

Hereinafter, embodiments of a deformed nail corrector according to the present invention will be described with reference to the accompanying drawings. The deformed nail corrector according to the present invention is intended to correct a deformed nail such as a pincer nail or an ingrown nail (particularly, a deformed toenail).

### First Embodiment

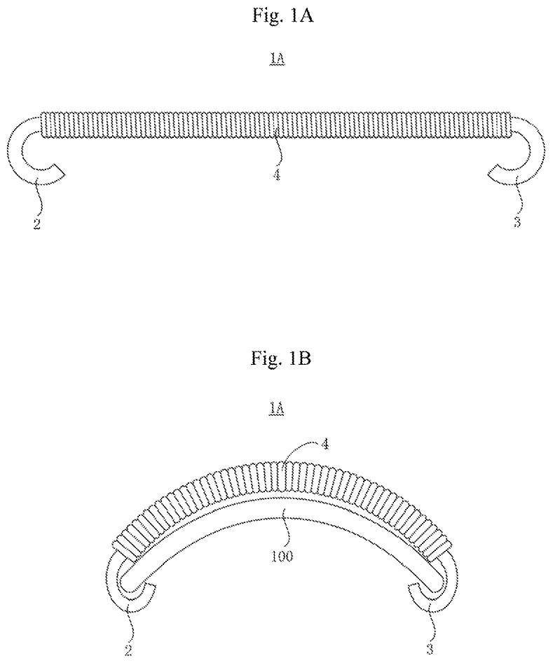

FIG. 1A illustrates a front view of a deformed nail corrector 1A according to a first embodiment of the present invention. As shown in the figure, the deformed nail corrector 1A includes a first hook 2, a second hook 3, and a coil 4 corresponding to the “flexible member” of the present invention.

FIG. 1B illustrates a front view of the deformed nail corrector 1A attached to a deformed nail 100. The deformed nail 100 shown in FIG. 1B is a toenail, but the toe is not shown. When attached to the deformed nail 100 (such a state will be referred to below as attached state), the deformed nail corrector 1A is bent along the deformed nail 100 so as to apply a corrective force.

The first hook 2 is attachable to a first edge of the deformed nail 100 in the width direction, as shown in FIG. 1B. Specifically, the first hook 2 is curved in a U-shape at a tip such that the tip can catch the underside of the deformed nail 100 at the first edge in the width direction. The first hook 2 is made of cylindrical stainless steel (e.g., SUS304).

The second hook 3 is attachable to a second edge of the deformed nail 100 in the width direction, as shown in FIG. 1B. Specifically, the second hook 3 is curved in a U-shape at a tip such that the tip can catch the underside of the deformed nail 100 at the second edge in the width direction. The second hook 3 is made of cylindrical stainless steel (e.g., SUS304).

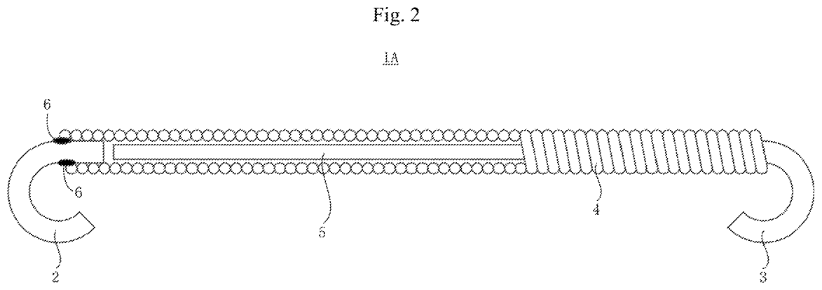

FIG. 2 illustrates the structure of the deformed nail corrector 1A. FIG. 2 shows an internal structure (cross-sectional view) on the first hook 2 side and an external structure (front view) on the second hook 3 side. As shown in the figure, the deformed nail corrector 1A includes a cylindrical wire member 5 slidably inserted in the coil 4. The wire member 5 is configured to apply a corrective force.

The coil 4 is joined to the first hook 2 with an end of the first hook 2 inserted at a first end, and also joined to the second hook 3 with an end of the second hook 3 inserted at a second end. In the present embodiment, the coil 4 has an inner surface joined at the first end to an outer surface at the end of the first hook 2 by laser beam welding (see a welded portion 6 in FIG. 2). Similarly, the coil 4 has an inner surface joined at the second end to an outer surface at the end of the second hook 3 by laser beam welding (not shown in the figure). The coil 4 is made of, for example, stainless steel (e.g., SUS304).

The coil 4 is flexible and stretchable. The coil 4 is stretchable such that the length from the first to the second end thereof, i.e., the distance between the first hook 2 and the second hook 3, can be changed. For example, when the first hook 2 is attached to the first edge of the deformed nail 100 in the width direction, if the second hook 3 is pulled, the coil 4 is stretched so that the distance between the first hook 2 and the second hook 3 increases. Accordingly, the second hook 3 can be readily attached to the second edge of the deformed nail 100 in the width direction. The coil 4 used in the present embodiment has a natural length of 17 mm.

The wire member 5 is superelastic and plays the role of correcting the deformed nail 100. Superelasticity refers to the properties that allow deformation caused by, for example, stress-induced martensitic transformation under load to be undone by reverse transformation upon unloading. In the present embodiment, the wire member 5 has a linear shape, and when the wire member 5 is bent in attached state, the wire member 5 tends to return to the linear shape. The deformed nail corrector 1A corrects the deformed nail 100 by utilizing such superelasticity. The wire member 5 is made of, for example, a Ni—Ti alloy.

Thus, since the deformed nail corrector 1A according to the present embodiment includes the coil 4, which is stretchable so as to change the distance between the first hook 2 and the second hook 3, the deformed nail corrector 1A can be readily attached to the deformed nail 100 by virtue of the stretchability of the coil 4.

### Second Embodiment

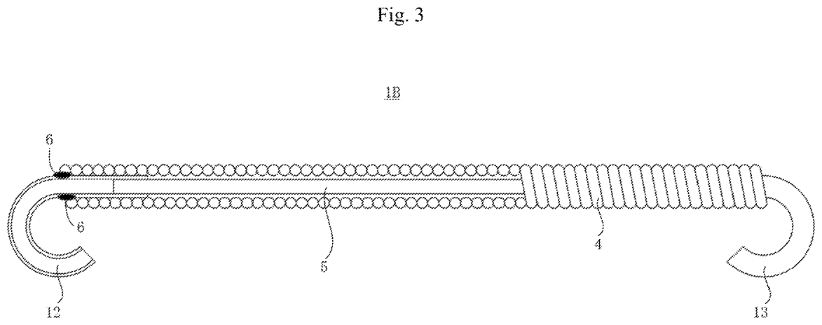

FIG. 3 illustrates a deformed nail corrector 1B according to a second embodiment of the present invention. As shown in the figure, the deformed nail corrector 1B includes a first hook 12, a second hook 13, a coil 4, and a wire member 5. The coil 4 and the wire member 5 are the same as those in the first embodiment and therefore will not be elaborated upon herein. Note that FIG. 3 shows an internal structure (cross-sectional view) on the first hook 12 side and an external structure (front view) on the second hook 13 side.

The first hook 12 is attachable to a first edge of a deformed nail 100 in the width direction. Specifically, the first hook 12 is curved in a U-shape at a tip such that the tip can catch the underside of the deformed nail 100 at the first edge in the width direction. The first hook 12 is in the shape of a circular tube having openings at the tip and the end and made of stainless steel (e.g., SUS304). The first hook 12 has a first end of the wire member 5 slidably inserted at the end thereof.

The second hook 13 is attachable to a second edge of the deformed nail 100 in the width direction. Specifically, the second hook 13 is curved in a U-shape at a tip such that the tip can catch the underside of the deformed nail 100 at the second edge in the width direction. The second hook 13 is in the shape of a circular tube having openings at the tip and the end and made of stainless steel (e.g., SUS304). The second hook 13 has a second end of the wire member 5 slidably inserted at the end thereof.

In attached state, the wire member 5 tends to return to the original linear shape, with the result that upward repulsive forces are created at the first and second ends of the wire member 5. In the present embodiment, since the first and second ends of the wire member 5 are respectively inserted in the first hook 12 and the second hook 13, the first hook 12 and the second hook 13 receive the repulsive forces. Thus, the deformed nail corrector 1B according to the present embodiment renders it possible to prevent the coil 4 from being inflected by the repulsive forces.

Furthermore, the deformed nail corrector 1B according to the present embodiment, as with that of the first embodiment, can be readily attached to the deformed nail 100 by virtue of the stretchability of the coil 4.

### Third Embodiment

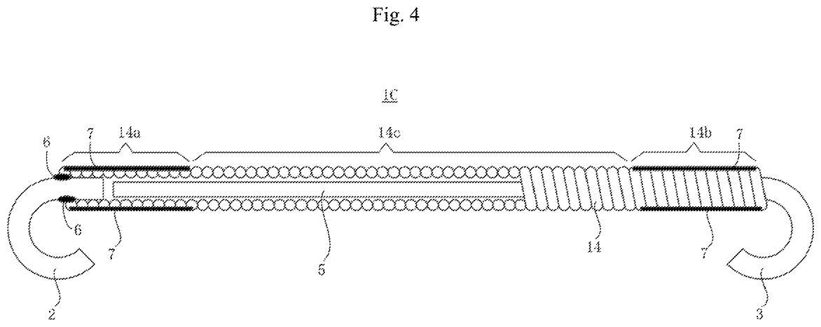

FIG. 4 illustrates a deformed nail corrector 1C according to a third embodiment of the present invention. As shown in the figure, the deformed nail corrector 1C includes a first hook 2, a second hook 3, a coil 14, and a wire member 5. The first hook 2, the second hook 3, and the wire member 5 are the same as those in the first embodiment and therefore will not be elaborated upon herein. Note that FIG. 4 shows an internal structure (cross-sectional view) on the first hook 2 side and an external structure (front view) on the second hook 3 side.

The coil 14 is joined to the first hook 2 with an end of the first hook 2 inserted at a first end, and also joined to the second hook 3 with an end of the second hook 3 inserted at a second end. In the present embodiment, the coil 14 has an inner surface joined at the first end to an outer surface at the end of the first hook 2 by laser beam welding (see a welded portion 6 in FIG. 4). Similarly, the coil 14 has an inner surface joined at the second end to an outer surface at the end of the second hook 3 by laser beam welding (not shown in the figure). The coil 14 is made of, for example, stainless steel (e.g., SUS304).

The coil 14 includes a first welded portion 14a on the first end side, a second welded portion 14b on the second end side, and an intermediate portion 14c. The first welded portion 14a includes two, upper and lower, welded parts formed on the outer surface by laser beam welding (see welded parts 7 in FIG. 4). The second welded portion 14b includes two, upper and lower, welded parts formed on the outer surface by laser beam welding (see welded parts 7 in FIG. 4). The first welded portion 14a and the second welded portion 14b are more rigid than the intermediate portion 14c and are neither flexible nor stretchable.

The wire member 5 is slidably inserted in the coil 14, and first and second ends of the wire member 5 are always positioned within the areas of the first and second welded portions 14a and 14b, respectively. Accordingly, in attached state, the first welded portion 14a and the second welded portion 14b receive repulsive forces created at the first and second ends of the wire member 5. Thus, the deformed nail corrector 1C according to the present embodiment renders it possible to prevent the coil 14 from being inflected by the repulsive forces.

The intermediate portion 14c of the coil 14 is flexible and stretchable. Specifically, the intermediate portion 14c is stretchable so as to allow the distance between the first hook 2 and the second hook 3 to be changed. For example, when the first hook 2 is attached to a first edge of a deformed nail 100 in the width direction, if the second hook 3 is pulled, the intermediate portion 14c is stretched so that the distance between the first hook 2 and the second hook 3 increases. Accordingly, the second hook 3 can be readily attached to a second edge of the deformed nail 100 in the width direction. Thus, the deformed nail corrector 1C according to the present embodiment can be readily attached to the deformed nail 100 by virtue of the stretchability of the intermediate portion 14c of the coil 14.

### Fourth Embodiment

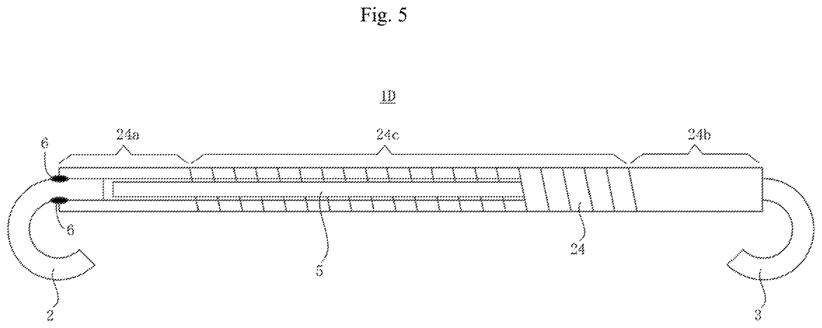

FIG. 5 illustrates a deformed nail corrector 1D according to a fourth embodiment of the present invention. As shown in the figure, the deformed nail corrector 1D includes a first hook 2, a second hook 3, a helical tube 24, and a wire member 5. The first hook 2, the second hook 3, and the wire member 5 are the same as those in the first embodiment and therefore will not be elaborated upon herein. Note that FIG. 5 shows an internal structure (cross-sectional view) on the first hook 2 side and an external structure (front view) on the second hook 3 side.

The helical tube 24 is joined to the first hook 2 with an end of the first hook 2 inserted at a first end, and also joined to the second hook 3 with an end of the second hook 3 inserted at a second end. In the present embodiment, the helical tube 24 has an inner surface joined at the first end to an outer surface at the end of the first hook 2 by laser beam welding (see a welded portion 6 in FIG. 5). Similarly, the helical tube 24 has an inner surface joined at the second end to an outer surface at the end of the second hook 3 by laser beam welding (not shown in the figure). The helical tube 24 is made of, for example, stainless steel (e.g., SUS304).

The helical tube 24 includes a first rigid portion 24a on the first end side, a second rigid portion 24b on the second end side, and an intermediate portion 24c. Only the intermediate portion 24c has a helical cut. The first rigid portion 24a and the second rigid portion 24b are more rigid than the intermediate portion 24c and are neither flexible nor stretchable.

The wire member 5 is slidably inserted in the helical tube 24, and first and second ends of the wire member 5 are always positioned within the areas of the first and second rigid portions 24a and 24b, respectively. Accordingly, in attached state, the first rigid portion 24a and the second rigid portion 24b receive repulsive forces created at the first and second ends of the wire member 5. Thus, the deformed nail corrector 1D according to the present embodiment renders it possible to prevent the helical tube 24 from being inflected by the repulsive forces.

The intermediate portion 24c of the helical tube 24 is flexible and stretchable. Specifically, the intermediate portion 24c is stretchable because of the helical cut, so that the distance between the first hook 2 and the second hook 3 can be changed. Thus, the deformed nail corrector 1D according to the present embodiment can be readily attached to a deformed nail 100 by virtue of the stretchability of the intermediate portion 24c of the helical tube 24.

### Fifth Embodiment

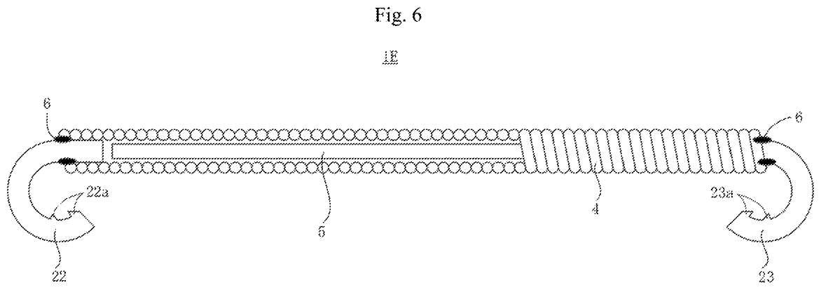

FIG. 6 illustrates a deformed nail corrector 1E according to a fifth embodiment of the present invention. As shown in the figure, the deformed nail corrector 1E includes a first hook 22, a second hook 23, a coil 4, and a wire member 5. The coil 4 and the wire member 5 are the same as those in the first embodiment and therefore will not be elaborated upon herein. Note that FIG. 6 shows an internal structure (cross-sectional view) on the first hook 22 side and an external structure (front view) on the second hook 23 side.

The first hook 22 has commonalities with the first hook 2 in the first embodiment, except that a barbed portion 22a is provided at a tip. The barbed portion 22a includes at least one barb (in the present embodiment, two barbs). The first hook 22 includes the barbed portion 22a and thereby achieves more reliable attachment to a deformed nail 100.

The second hook 23 has commonalities with the second hook 3 in the first embodiment, except that a barbed portion 23a is provided at a tip. The barbed portion 23a includes at least one barb (in the present embodiment, two barbs). The second hook 23 includes the barbed portion 23a and thereby achieves more reliable attachment to the deformed nail 100.

Thus, the deformed nail corrector 1E according to the present embodiment includes the barbed portions 22a and 23a and thereby achieves more reliable attachment to the deformed nail 100. Note that the barbed portions 22a and 23a can also be applied to all other embodiments.

### Sixth Embodiment

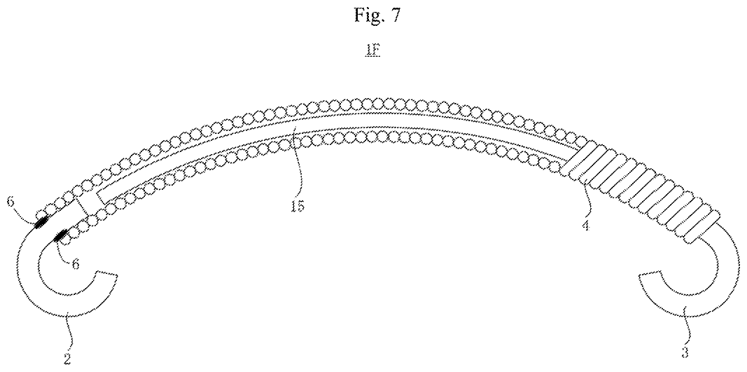

FIG. 7 illustrates a deformed nail corrector 1F according to a sixth embodiment of the present invention. As shown in the figure, the deformed nail corrector 1F includes a first hook 2, a second hook 3, a coil 4, and a wire member 15. The first hook 2, the second hook 3, and the coil 4 are the same as those in the first embodiment and therefore will not be elaborated upon herein. Note that FIG. 7 shows an internal structure (cross-sectional view) on the first hook 2 side and an external structure (front view) on the second hook 3 side.

The wire member 15 is made of, for example, a cylindrical Ni—Ti alloy and is superelastic. In the present embodiment, given that a normal human nail is arc-like, the wire member 15 is formed to be arc-like. When the wire member 15 is bent in attached state, the wire member 15 tends to return to the arc-like shape. Accordingly, when compared to the wire member 5, which tends to return to a linear shape, the wire member 15 creates a weaker corrective force. Therefore, the deformed nail corrector 1F according to the present embodiment renders it possible to prevent a deformed nail 100 from being excessively corrected (e.g., being bent backwards). Note that the wire member 15 can also be applied to all other embodiments.

The coil 4 is flexible and therefore becomes arc-like when the wire member 15 is inserted therein. The coil 4 does not lose stretchability even when the coil 4 becomes arc-like. Thus, the deformed nail corrector 1F according to the present embodiment can be readily attached to the deformed nail 100 by virtue of the stretchability of the coil 4.

### Seventh Embodiment

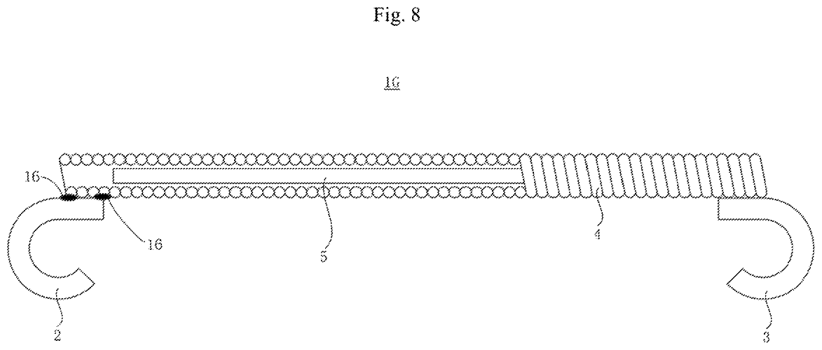

FIG. 8 illustrates a deformed nail corrector 1G according to a seventh embodiment of the present invention. As shown in the figure, the deformed nail corrector 1G includes a first hook 2, a second hook 3, a coil 4, and a wire member 5. FIG. 8 shows an internal structure (cross-sectional view) on the first hook 2 side and an external structure (front view) on the second hook 3 side.

The deformed nail corrector 1G according to the present embodiment is the same as that in the first embodiment in terms of joining relationships, except for the joining relationships of the coil 4 with the first hook 2 and the second hook 3. In the present embodiment, the coil 4 has an outer surface joined at a first end to an outer surface at an end of the first hook 2 by laser beam welding (see welded portions 16 in FIG. 8). Similarly, the coil 4 has an outer surface joined at a second end to an outer surface of an end of the second hook 3 by laser beam welding (not shown in the figure).

In attached state, the coil 4 is positioned above the first hook 2 and the second hook 3. Moreover, the coil 4 has uncovered openings at the first and second ends. Accordingly, when the deformed nail corrector 1G according to the present embodiment is in attached state, the wire member 5 can be put in and out of the coil 4 from any of the first and second ends.

For example, inserting a wire member 5 having a smaller outer diameter into the coil 4 results in a decreased corrective force, whereas inserting a wire member 5 having a larger outer diameter into the coil 4 results in an increased corrective force. That is, the deformed nail corrector 1G according to the present embodiment renders it possible to adjust the corrective force in attached state. Note that this configuration (the joining relationships of the coil 4 with the first hook 2 and the second hook 3) can also be applied to all other embodiments.

Furthermore, the deformed nail corrector 1G according to the present embodiment, as with that of the first embodiment, can be readily attached to a deformed nail 100 by virtue of the stretchability of the coil 4.

### Eighth Embodiment

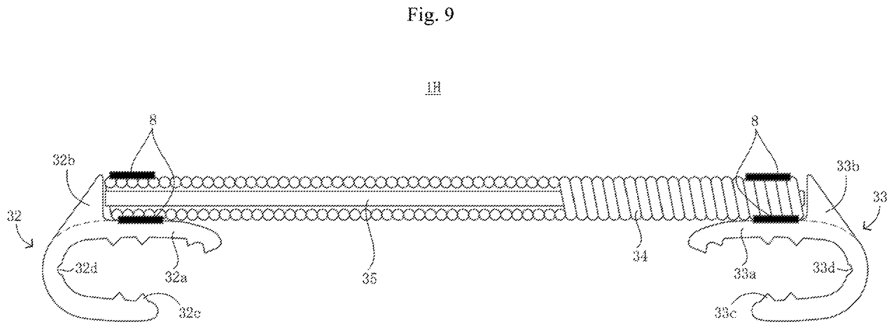

FIG. 9 illustrates a deformed nail corrector 1H according to an eighth embodiment of the present invention. As shown in the figure, the deformed nail corrector 1H includes a first hook 32, a second hook 33, a coil 34, and a wire member 35. FIG. 9 shows an internal structure (cross-sectional view) on the first hook 32 side and an external structure (front view) on the second hook 33 side.

The first hook 32 is made of, for example, stainless steel (e.g., SUS304) and provided at a first end of the coil 34. The first hook 32 has beveled circumferential edges. The first hook 32 is constituted by a main hook portion 32a, which is a plate-like portion curved with an approximately U-shaped cross section, and an extended portion 32b formed on a top surface of the main hook portion 32a. The first hook 32 is slightly wider than the outer diameter of the coil 34.

The main hook portion 32a includes a plurality of barbs 32c provided on a bottom surface of an upper part and a top surface of a lower part (in the present embodiment, four on the upper part and two on the lower part); the upper and lower parts are to be positioned on the topside and underside, respectively, of a deformed nail 100 at a first edge in the width direction of the deformed nail 100. The barbs 32c are formed to extend in the width direction of the main hook portion. The barbs 32c, as with the barbed portions 22a and 23a in FIG. 6, are designed to achieve more reliable attachment to the deformed nail 100. Note that the number and the shape of the barbs 32c can be suitably changed.

The gap between the upper part of the main hook portion 32a and a bottom portion of the coil 34 increases away from the first end of the coil 34 (i.e., toward the intermediate portion of the coil 34). Moreover, the curved part of the main hook portion 32a has a recess 32d extending widthwise across the center of an inside surface.

The extended portion 32b has a first surface formed approximately in the shape of a triangle when viewed from the front, and extending upward from the top surface of the main hook portion 32a (in the present embodiment, the first surface is a vertical plane and therefore stands upright). The first surface is opposed to an opening at the first end of the coil 34 so as to keep the wire member 35 from falling from the first end of the coil 34.

The second hook 33 is made of, for example, stainless steel (e.g., SUS304) and provided at a second end of the coil 34. The second hook 33 has beveled circumferential edges. Similar to the first hook 32, the second hook 33 is constituted by a main hook portion 33a, which is a plate-like portion curved with an approximately U-shaped cross section, and an extended portion 33b formed on a top surface of the main hook portion 33a. The second hook 33 has the same width as the first hook 32.

The main hook portion 33a includes a plurality of barbs 33c provided on a bottom surface of an upper part and a top surface of a lower part (in the present embodiment, four on the upper part and two on the lower part); the upper and lower parts are to be positioned on the topside and underside, respectively, of the deformed nail 100 at a second edge in the width direction of the deformed nail 100. The barbs 33c are formed to extend in the width direction of the main hook portion. The barbs 33c, as with the barbed portions 22a and 23a in FIG. 6, are designed to achieve more reliable attachment to the deformed nail 100. Note that the number and the shape of the barbs 33c can be suitably changed.

The gap between the upper part of the main hook portion 33a and the bottom portion of the coil 34 increases away from the second end of the coil 34 (i.e., toward the intermediate portion of the coil 34). Moreover, the curved part of the main hook portion 33a has a recess 33d extending widthwise across the center of an inside surface.

The extended portion 33b has a second surface formed approximately in the shape of a triangle when viewed from the front, and extending upward from the top surface of the main hook portion 33a (in the present embodiment, the second surface is a vertical plane and therefore stands upright). The second surface is opposed to an opening at the second end of the coil 34 so as to keep the wire member 35 from falling from the second end of the coil 34.

The coil 34 is made of stainless steel (e.g., SUS304) or a cobalt-base alloy. The coil 34, enclosing the wire member 35, has the bottom portion joined at the first end to the top surface of the main hook portion 32a of the first hook 32 and also at the second end to the top surface of the main hook portion 33a of the second hook 33. In the present embodiment, the coil 34 is joined to the first hook 32 and the second hook 33 by laser cladding in which laser beam welding is performed while a predetermined metal is being supplied (see cladded portions 8 in FIG. 9).

The coil 34 also has cladded portions 8 formed on the top at the first and second ends by laser cladding. The coil 34 is neither flexible nor stretchable at the portions (first and second end portions) where the cladded portions 8 are formed, but is flexible and stretchable at the portion (intermediate portion) where no cladded portions 8 are formed. The first and second end portions of the coil 34 are more rigid and thicker than the intermediate portion.

The wire member 35 is superelastic and plays the role of correcting the deformed nail 100. The wire member 35 has a linear shape and tends to return to the linear shape when bent in attached state. The deformed nail corrector 1H corrects the deformed nail 100 by virtue of such superelasticity. The wire member 35 is made of, for example, a Ni—Ti alloy. The wire member 35 has the same length as the natural length of the coil 34 (in the present embodiment, 18 mm) but may be shorter than the natural length of the coil 34.

### Ninth Embodiment

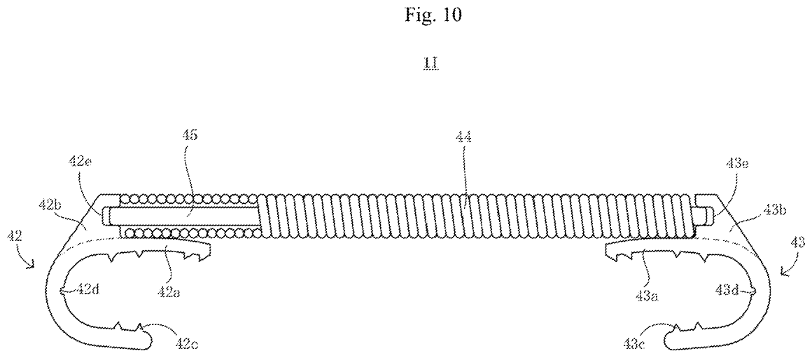

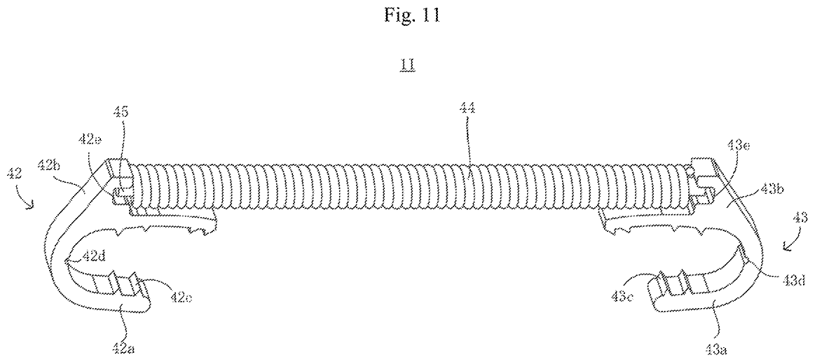

FIGS. 10 and 11 illustrate a deformed nail corrector 1I according to a ninth embodiment of the present invention. As shown in the figures, the deformed nail corrector 1I includes a first hook 42, a second hook 43, a coil 44, and a wire member 45. FIG. 10 shows an internal structure (cross-sectional view) on the first hook 42 side and an external structure (front view) on the second hook 43 side.

The coil 44 and the wire member 45 are the same as the coil 34 and the wire member 35 in the eighth embodiment, except that the wire member 45 is longer than the natural length of the coil 44. Moreover, the coil 44, as with that of the eighth embodiment, is joined to the first hook 42 and the second hook 43 by laser cladding. In FIGS. 10 and 11, cladded portions 8 formed at first and second end portions of the coil 44 are omitted.

The first hook 42 is constituted by a main hook portion 42a, which is a plate-like portion curved with an approximately U-shaped cross section, and an extended portion 42b formed on a top surface of the main hook portion 42a. The first hook 42 has beveled circumferential edges. The main hook portion 42a is the same as the main hook portion 32a in the eighth embodiment and therefore will not be elaborated upon herein.

The extended portion 42b has a first surface opposed to an opening at a first end of the coil 44, and specifically, the first surface extends upward from the top surface of the main hook portion 42a. The first surface has formed therein a first groove 42e in which to accommodate a first end of the wire member 45. In attached state, the first groove 42e receives a repulsive force created at the first end of the wire member 45. Thus, the coil 44 can be prevented from being damaged or inflected by the repulsive force. In the present embodiment, the first groove 42e is formed across the width, but the first groove 42e may be formed across a portion of the width.

The second hook 43 is constituted by a main hook portion 43a, which is a plate-like portion curved with an approximately U-shaped cross section, and an extended portion 43b formed on a top surface of the main hook portion 43a. The second hook 43 has beveled circumferential edges. The main hook portion 43a is the same as the main hook portion 33a in the eighth embodiment and therefore will not be elaborated upon herein.

The extended portion 43b has a second surface opposed to an opening at a second end of the coil 44, and specifically, the second surface extends upward from a top surface of the main hook portion 43a. The second surface has formed therein a second groove 43e in which to accommodate a second end of the wire member 45. In attached state, the second groove 43e receives a repulsive force created at the second end of the wire member 45. Thus, the coil 44 can be prevented from being damaged or inflected by the repulsive force. In the present embodiment, the second groove 43e is formed across the width, but the second groove 43e may be formed across a portion of the width.

While the embodiments of the deformed nail corrector according to the present invention have been described above, the present invention is not limited to the embodiments.

The shape, material, etc., of the first hook of the present invention can be suitably changed, so long as the first hook is attachable to the first edge of the deformed nail 100 in the width direction. Similarly, the shape, material, etc., of the second hook of the present invention can be suitably changed, so long as the second hook is attachable to the second edge of the deformed nail 100 in the width direction.

As the flexible member of the present invention, any element other than a coil or a helical tube may also be used, so long as the element is in the shape of a tube in which the wire member of the present invention can be inserted, and the distance between the first and second hooks can be changed by the element being stretched with the first and second ends respectively being joined to the first and second hooks of the present invention. Moreover, the material of the coil is preferably stainless steel (e.g., SUS304) or a cobalt-base alloy but can be suitably changed, so long as the coil is flexible and stretchable.

Any method other than laser beam welding can also be applied to joining the flexible member to the first and second hooks. For example, methods involving pressure bonding, brazing, resin adhesives, etc., can be applied.

The shape, material, etc., of the wire member of the present invention can be suitably changed, so long as the wire member is superelastic and can be inserted in the flexible member. For example, superelastic metals such as a Ni—Ti—Co alloy and a Ni—Ti—Fe alloy may be used. Alternatively, high-strength, high-toughness engineering plastics may be used.

What is claimed is:

1. A deformed nail corrector for correcting a deformed nail, comprising:
a first hook attachable to a first edge of the deformed nail in a width direction;
a second hook attachable to a second edge of the deformed nail in the width direction;
a tube-like flexible member joined at a first end to the first hook and at a second end to the second hook; and
a superelastic wire member inserted in the flexible member, wherein,
the flexible member is stretchable so as to allow a distance between the first hook and the second hook to be changed.

2. The deformed nail corrector according to claim 1, wherein the flexible member is joined to the first hook having an end inserted from the first end of the flexible member, and also joined to the second hook having an end inserted from the second end of the flexible member.

3. The deformed nail corrector according to claim 2, wherein,
the first hook has a hollow shape with an opening at the end,
the second hook has a hollow shape with an opening at the end, and
the wire member has a first end slidably inserted in the first hook and a second end slidably inserted in the second hook.

4. The deformed nail corrector according to claim 2, wherein,
the flexible member is a coil including a first welded portion, a second welded portion, and an intermediate portion between the first welded portion and the second welded portion, the first welded portion having a welded outer surface at the first end, and the second welded portion having a welded outer surface at the second end,
the first welded portion and the second welded portion are more rigid than the intermediate portion, and
the wire member has a first end positioned within an area of the first welded portion and a second end positioned within an area of the second welded portion.

5. The deformed nail corrector according to claim 2, wherein,
the flexible member is a helical tube including a first rigid portion at the first end, a second rigid portion at the second end, and an intermediate portion with a helical cut,
the first rigid portion and the second rigid portion are more rigid than the intermediate portion, and
the wire member has a first end positioned within an area of the first rigid portion and a second end positioned within an area of the second rigid portion.

6. The deformed nail corrector according to claim 1, wherein the first hook and the second hook include barbed portions at tips.

7. The deformed nail corrector according to claim 1, wherein the wire member has an arc-like shape and tends to return to the arc-like shape when deformed.

8. The deformed nail corrector according to claim 1, wherein the flexible member has an outer surface joined to the first hook at the first end and to the second hook at the second end, so as to allow the wire member to be put in and out of the flexible member from either the first or second end, or both.

9. The deformed nail corrector according to claim 1, wherein,
the first hook has a first main hook portion and a first extended portion formed on a top surface of the first main hook portion,
the first extended portion has a first surface extending upward from the top surface,
the second hook has a second main hook portion and a second extended portion formed on a top surface of the second main hook portion,
the second extended portion has a second surface extending upward from the top surface, and
the flexible member has a bottom portion joined at the first end to the top surface of the first main hook portion and at the second end to the top surface of the second main hook portion, such that the opening at the first end is opposed to the first surface and the opening at the second end is opposed to the second surface.

10. The deformed nail corrector according to claim 9, wherein,
the wire member is longer than a natural length of the flexible member,
the first extended portion has a first groove formed in the first surface so as to accommodate a first end of the wire member, and
the second extended portion has a second groove formed in the second surface so as to accommodate a second end of the wire member.
